(19) United States
(12) Patent Application Publication
(10) Pub. No.: US 2005/0176693 A1
(43) Pub. Date: Aug. 11, 2005

(54) Method of intermittent administration of a pharmaceutical for the treatment of conditions associated with a female's menstrual cycle

(76) Inventors: Roger M. Boissonneault, Long Valley, NJ (US); Tina M. deVries, Long Valley, NJ (US); Herman Ellman, Boonton Township, NJ (US); Kathryn L. MacFarlane, Morristown, NJ (US); Kelly F. Smith, Mountain Lakes, NJ (US)

Correspondence Address: FITZPATRICK CELLA HARPER & SCINTO, 30 ROCKEFELLER PLAZA, NEW YORK, NY, 10112, US

(21) Appl. No.: 11/057,843

(22) Filed: Feb. 15, 2005

Related U.S. Application Data
(63) Continuation-in-part of application No. 10/819,764, filed on Apr. 7, 2004.
Application No. 10/819,764 is a continuation-in-part of application No. 10/762,263, filed on Jan. 23, 2004, now abandoned.
Application No. 10/762,263 is a continuation-in-part of application No. 10/639,891, filed on Aug. 12, 2003, now abandoned.

(51) Int. Cl. A61K 31/57; A61K 31/343; A61K 31/137

(52) U.S. Cl. 514/171; 514/469; 514/649

(57) ABSTRACT

The present invention is a method of non-continuous administration of a pharmaceutical to a female for a condition associated with the female's menstrual cycle, comprising administering a daily placebo dosage, starting on the first or the second day of the menstrual cycle and continuing the daily administration for a placebo dosage period. Next administering a daily pharmaceutical dosage, starting on the day after the last daily placebo dosage was administered and continuing the daily administration of the pharmaceutical dosage for a pharmaceutical dosage period. Both the placebo dosages and the pharmaceutical dosages are contained in a single package.

### REFERENCE TO RELATED APPLICATION

This application is a continuation-in-part of U.S. patent application Ser. No. 10/819,764, filed Apr. 7, 2004, which is a continuation-in-part of U.S. patent application Ser. No. 10/762,263, filed Jan. 23, 2004, which is a continuation in-part of U.S. patent application Ser. No. 10/639,891, filed Aug. 12, 2003.

### BACKGROUND OF THE INVENTION

1. Field of the Invention

This invention is directed to a method of intermittent administration of a pharmaceutical to a woman for the treatment of cyclical physical and, or, psychological symptoms that are associated with the menstrual cycle. The invention is also directed to packaging used in the method of administration.

2. Related Background Art

Any number of conditions in women occur intermittently in association with the menstrual cycle. These conditions include, for example, migraine headaches, exacerbation of pain due to endometriosis, and PMDD. A detailed discussion of an example follows: Each month, for a few days prior to the onset of menstruation, many millions of women experience menstrual cycle physical and psychological symptoms, which if found to be repetitive are generally called premenstrual syndrome (PMS). Symptoms of PMS commonly include changes in mood and appetite, which may include becoming saddened, tearful, irritable, angry, anxious, feeling hopelessness or having carbohydrate cravings. In addition, these women suffering from PMS commonly have cyclical physical symptoms, which may include breast tenderness, swelling, headaches, joint or muscle pain, bloating or weight gain.

A severe, predominantly psychological form of the more general PMS is known as premenstrual dysphoric disorder (PMDD), in which general symptoms of PMS markedly interfere with social activities and relationships with others, including at home or work. The Diagnostic and Statistical Manual of Mental Disorders, 4th Edition. Previously PMDD was known as late luteal phase dysphoric disorder (LLPDD). The Diagnostic and Statistical Manual of Mental Disorders, 3rd Edition. PMS, PMDD and LLPDD are used herein interchangeably.

There have been numerous suggestions made about the cause of PMS. For example, some hypothesized that it was caused by a uterine toxin. Others suggested its cause was over consumption of sweets, which was presumably followed by excessive insulin secretion, hypoglycemia, and inadequate brain glucose and resulted in the often observed depression and anxiety. It has also been postulated that the behavioral symptoms result from the tissue edema often observed and that the psychological changes result from feelings of loss or the social complexities generated by the discomforts of menstruation. Some have speculated that progesterone metabolites are implicated.

However, none of these theories has been substantiated: PMS/PMDD can persist after hysterectomy and, hence, uterine toxins cannot be its cause; the hyperinsulinism of PMS/PMDD is not associated with low blood glucose levels, and is probably the consequence of a behavioral aberration (i.e., the tendency of premenstrual women to choose high-carbohydrate diets, which potentiate insulin secretion)—rather than the cause; the mood and appetitive changes of PMS/PMDD are poorly correlated with the tissue swelling; and subhuman primates who are presumably exempt from the psychodynamic or social complexities of human life, also exhibit characteristic behavioral changes premenstrually.

There have been many treatments suggested for overcoming or reducing the symptoms of PMS/PMDD. These include carbohydrate-free diets, reducing salt, alcohol and caffeine intake, vitamin supplements, ovarian hormones, detoxifying agents, irradiation of the ovaries and pituitary, use of diuretics, use of antimineralocorticoids (e.g. aldactone) and use of oral contraceptives.

Calcium has been found to be an effective treatment for alleviating some of the symptoms associated with PMS. It has been especially effective in treating pain and water retention. S. Thys-jacobs, S. Ceccarelli, A. Beirman, H. Weisman, M. A. Cohen and J. Alvir, Calcium supplementation in premenstrual syndrome: A randomized crossover trial, J. Gen. Int. Med. 4: 183-189, (1989).

Magnesium supplementation has been shown to relieve some symptoms of PMS, particularly pain and mood fluctuations. F. Fancchinetti, P. Borella, G. Sances, L. Fioroni, R. Nappi and A. R. Genazzani, Oral Magnesium Successfully Relieves Premenstrual Mood Changes, Obstetrics & Gynecology, 78: 177-181, (1991).

U.S. Pat. No. 5,354,743 discloses that some women suffering from symptoms associated with PMS have lower levels of 25 hydroxyvitamin D compared to asymptomatic women. Thus, suggesting that vitamin D could be used to alleviate some of the symptoms associated with PMS. Furthermore, vitamin D may be useful in combination with calcium and magnesium supplementation since it is known that vitamin D increases the absorption of calcium and magnesium in the human body and is also involved with the transportation of calcium through cell membranes.

Diuretics have been used to relieve the symptoms related to water retention such as bloating, swelling, painful breasts, cramps and tension. Common diuretics are pamabrom, ammonium chloride, hydrochlorothiazide, and spironolactone.

Nonprescription pharmacological compositions have also been used. These compositions generally contain an analgesic, such as aspirin, acetaminophen or ibuprophen, for the treatment of back pain, headache pain and abdominal cramping. Sometimes these compositions will also contain an antispasmodic to help treat the abdominal cramping.

U.S. Pat. No. 4,897,411 discloses the use of clonidine to treat the symptoms associated with PMS. Clonidine is a centrally acting antihypertensive agent.

U.S. Pat. No. 4,971,998 discloses that administration of d-fenfluramine and fluoxetine, which selectively enhances serotonin-mediated neurotransmission, are useful to treat PMS and LLPDD. Agents or drugs useful in enhancing serotonin-mediated neurotransmission, or the effect of serotonin within the brain synapses, are referred to as serotoninergic drugs and include (1) drugs which act to increase the quantity of serotonin present within the synapses and (2) drugs which act to enhance the effects of serotonin present with brain synapses, generally by activating post-synaptic serotonin receptors.

The neurotransmitter serotonin (5-hydroxytryptamine or 5-HT) is 3-(beta-aminoethyl)-5-hydroxyindole. It stimulates or inhibits a variety of smooth muscles and nerves and, among others, has effects on secretion by both exocrine and endocrine glands and on functioning of the respiratory, cardiovascular and central nervous systems. Within the central nervous system (CNS), serotonin serves as a neurotransmitter in the brain and spinal cord, where it is the chemical transmitter of neurons referred to as tryptaminergic or serotoninergic neurons. These neurons are involved in control of sleep, appetite, nutrient selection, blood pressure, mood, endocrine secretion, aggressivity and numerous other sensitivities to external stimuli.

Administration of fluoxetine, which suppresses reuptake of serotonin and, thus, increases the quantity of serotonin available at brain synapses, has been shown to ameliorate the symptoms of PMS/PMDD. Steiner et al., New England Journal of Medicine, 232: 1529-34 (1995). Intermittent administration of fluoxetine for approximately fourteen days prior to the start of menses has also been shown to be efficacious in treating the symptoms of PMS. Cohen et al., Obstet & Gynecol., 100: 435-44 (2000). This suggests that other serotonin reuptake inhibitors would likewise be efficacious in the treatment of the symptoms associated with PMS.

Frequently patients start administration of fluoxetine about 14 days prior to menstruation. Since menstruation generally occurs in 28 day cycles, a good way to determine when to start the administration is to wait 14 days after the prior menstruation cycle has begun. However, this strategy causes difficulty in compliance because it requires the patient keep track of when her cycle has begun and then remember to start taking her medication 14 days later.

The symptoms associated with PMS can be treated with intermittent dosing, which may lessen the likelihood of side effects. Specifically, intermittent dosing may be less likely to cause reduced libido, insomnia and/or anxiety.

A menstrual cycle's length can range between 24 to 33 days in >95% of women with regular cycles, with 28 days being the average length. Thus, any method of treatment for the symptoms of PMS will have to contend with the fact that a women's menstrual cycle can vary in length.

A method of intermittent administration of a pharmaceutical to a woman for the treatment of cyclical physical and, or, psychological symptoms that are associated with the luteal phase of the menstrual cycle commonly referred to as premenstrual syndrome would be highly desirable. In addition, a method of packaging the pharmaceutical to assist the patient in following this method of administration, and thereby, remaining compliant with the treatment would be advantageous.

### SUMMARY OF THE INVENTION

The present invention is a method of non-continuous administration of a pharmaceutical to a human female for a condition associated with the female's menstrual cycle.

More specifically, a first embodiment of this invention is directed to a method of non-continuous administration of a pharmaceutical to a human female for a condition associated with the luteal phase of a female's menstrual cycle, comprising the steps of: a) orally administering a daily placebo dosage, starting on the first day of the female's menstrual cycle or the subsequent day thereof and continuing the daily oral administration of the daily placebo dosage for a placebo dosage period, and (b) orally administering a daily dosage of the pharmaceutical starting on the day after the last daily placebo dosage was administered and continuing the daily oral administration of the daily dosage of the pharmaceutical for the earlier of (i) a pharmaceutical dosage period or (ii) the onset of menses. The pharmaceutical dosage period is an effective number of days to treat the condition, e.g. 5 to 21 days and the placebo dosage period is generally in a range of 7 to 23 days in length. There is no need for a female to ascertain the number of days in her menstrual cycle and calculate the number of placebo dosages that should be administered based on the ascertained length of her menstrual cycle using the method of this invention. In a preferred embodiment, the pharmaceutical dosage is fluoxetine or a pharmaceutically acceptable salt thereof, and in a particularly preferred embodiment 15 to 18 days of fluoxetine or a pharmaceutical acceptable salt thereof are administered after the administration of 13 days of placebo. The invention also includes a package having a daily allotment of placebo dosages and fluoxetine dosages for practicing this embodiment of the method of this invention.

The oral administration of the daily placebo dosage begins on the first day or the second day of the menstrual cycle. The flexibility in starting the daily dosage makes it more convenient for the user because menstruation can start at any time of the day and therefore it may be easier to be certain of administering the daily placebo dosage on the second day following the start of menstruation. On the other hand, it may be equally convenient to simply have the user take both the pharmaceutical dosage from the package in use and the daily placebo dosage from a new package when the onset of menses occurs on a day in which the user has already administered a pharmaceutical dosage.

The present invention will assist the patient in complying with the treatment. It is known that patients become more readily acclimated to a daily dosage regimen than to an intermittent dosing regimen. Through the use of placebo dosages in conjunction with the pharmaceutical dosages, the patient will be administering a dosage every day and thus, this routine will become part of their normal daily activity. It also alleviates the need for the patient to keep track of the elapsed days in her menstrual cycle, this function being performed by the placebo dosages. Thus, a patient using this method of administering a pharmaceutical for the treatment of one or more symptoms associated with her menstrual cycle, such as the treatment of PMS/PMDD, should have a higher degree of compliance, a higher degree of accurate luteal phase dosing, and therefore a higher likelihood of efficacious dosing than one who is required to count off elapsed days in her menstrual cycle in order to intermittently administer the necessary dosages.

### BRIEF DESCRIPTION OF THE DRAWING

FIGS. 1-2 illustrate exemplary packages of this invention for practicing the method of the invention.

### DETAILED DESCRIPTION

This invention contemplates the use of any pharmaceutical, i.e., a substance, used in the treatment of a disease, an illness, or the symptoms of a disease, illness or physiological condition associated with a female's menstrual cycle. Exemplary pharmaceuticals employed in the present invention include selective serotonin reuptake inhibitors, non-steroidal anti-inflammatory drugs (NSAIDs), serotonin 5HT-1 antagonists, retinoids, antibiotics, analgesics, diuretics, and corticosteroids. Fluoxetine is a particularly preferred selective serotonin uptake inhibitor.

Exemplary conditions which may be associated with a female's menstrual cycle include premenstrual syndrome, migraine headache, endometriosis, psoriasis, acne, dysmenorrhea, neurosia, asthma and premenstrual cramps.

As used herein, the phrase “daily placebo dosage” refers to the entire amount of the placebo to be administered to the patient in one day. The entire amount may be in a single dosage unit, or may be divided into a number of separate units to be administered at different time periods during the course of the day.

As used herein, the phrase “daily pharmaceutical dosage” refers to the entire amount of a pharmaceutical to be administered to the patient in one day. The entire amount may be in a single dosage unit, or may be divided into a number of separate units to be administered at different time periods during the course of the day.

As used herein, the term “fluoxetine” refers to the following compound: 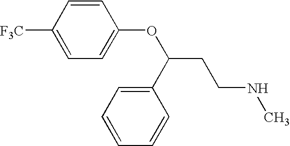
or a pharmaceutically acceptable salt thereof. The specific dose level of fluoxetine for any particular patient will depend upon a variety of factors, including the patient's age, body weight and the severity of the symptoms. Typically this amount would be between about 0.01 to about 300 mg/kg of body weight, with a preferred amount being between about 5 to about 60 mg/day more preferably about 10 to about 20 mg/day. A skilled artisan could routinely determine the proper amount for an individual patient.

As used herein, the phrase “pharmaceutically acceptable salt” refers to a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, g-hydroxybutyrates, glycollates, tartrates, methane-sulfonates (mesylates), propanesulfonates, naphthalene-1-sulfonates, naphthalene-2-sulfonates, and mandelates. A desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid or ethanesulfonic acid, or the like. In the present invention the hydrochloride salt is the preferred salt.

As used herein, the term “placebo” refers to a dosage form that does not contain a therapeutically effective amount of a pharmaceutical. The placebo could contain either one or more vitamins and, or, minerals. Preferably the placebo is an inert medicament which has no therapeutic value. In the present invention the placebo dosages are administered to the patient on days when the pharmaceutical is not required to be administered to treat the symptoms associated with a female's menstrual cycle, such as PMS. The effect is that the patient must administer a dosage, of either the pharmaceutical or a placebo, every day. The repetition of daily administration will eventually become part of the patient's every day routine, thereby increasing the likelihood of compliance. The placebo dosages in the present invention also eliminate the need for the patient to keep track of the elapsed days after the first day of her menstrual cycle.

As used herein, the phrase “single dosage unit” is used to indicate that the entire daily dosage is contained in one tablet, capsule (e.g., “PULVULES” a registered trademark of Eli Lilly and Company), caplet, or any other vehicle commonly known by those skilled in the art.

The number of days in a female's menstrual cycle is often known by the female. For some females, the menstrual cycle varies in length. A menstrual cycle can vary in length between 24 to 33 days in 95% of women with regular cycles, wherein the average length is 28 days. As previously noted, the method of this invention may be practiced without ascertaining the number of days in a female's menstrual cycle because the placebo dosage period may be fixed based on the average menstrual cycle of a female and the number of days the pharmaceutical is to be given so as to be effective to treat the condition.

Administration of the daily placebo may be dosage started on the first day of the menstrual cycle, i.e., the first day of menstruation, or alternatively, administration of the first dosage may be started on the second day of the menstrual cycle, i.e., the day after menstruation begins.

This pharmaceutical dosage period is selected to achieve effective treatment of the condition that is being treated. The placebo dosage period may be arrived at for a particular pharmaceutical by substracting the number of treatment days from the average number of days in a female's menstrual cycle, e.g., days of placebo equals twenty eight days minus days of pharmaceutical for effective treatment.

It may be preferable upon completion of the pharmaceutical dosage period to continue the daily oral administration of the pharmaceutical daily dosage until the first of, exhaustion of the pharmaceutical in the package or the start of menses. This optional embodiment may be useful when the human female's menstrual cycle exceeds the average female's menstrual cycle. It should be apparent in this case that the number of days that the pharmaceutical will be administered may exceed the number of days needed to achieve effective treatment of the condition that is being treated. For example, while the selected number of days to achieve effective treatment for a given pharmaceutical might be 15 days, a package could contain a greater number of daily dosages of the pharmaceutical, e.g. 18 days, to provide for additional daily dosages of the pharmaceutical for those instances when the female's menstrual cycle was longer than the average cycle.

Typically the pharmaceutical is administered using the method of the invention for 5 to 21 days, more preferably for 5 to 15 days and even more preferably 15 days with additional daily dosages up to 20, more preferably 18, days if the human female has a longer than average cycle, e.g. greater than 28 days. It is important to note that the placebos and pharmaceutical dosages used in the present invention are provided in a single package.

In a more specific embodiment of the method of the present invention, the preferred condition of treatment is premenstrual syndrome. In a more preferred embodiment of this method the pharmaceutical is a selective serotonin reuptake inhibitor. A more specific preferred embodiment is where the selective serotonin reuptake inhibitor is selected from the group consisting of citalopram, escitalopram, fluvoxamine, paroxetine, fluoxetine, sertraline, duloxetine and pharmaceutically acceptable salts thereof. In the most preferred embodiment the selective serotonin reuptake inhibitor is fluoxetine or a pharmaceutically acceptable salt thereof.

This method of treating PMS with fluoxetine differs from treatment with oral contraceptives. Both oral contraceptives and the present invention may be packaged in calendar or dial packs. Oral contraceptives, however, entrain the menstrual cycle, and therefore, do not have to contend with cycle lengths other than 28 days. Where as the present invention is designed to accommodate any women's menstrual cycle, regardless of the length.

The present invention also allows for at least a 50% dosage reduction in the average woman with PMS compared to a continuous dosing regimen. Thus, the present pharmaceutical regimen not only allows for ease of compliance, but may be safer.

The pharmaceutical dosage period is an effective number of days to treat the condition and ranges from 5 to 21 days, while the placebo dosage period will typically range from 7 to 23 days. In a preferred embodiment, while the effective pharmaceutical dosage period may be determined to be 15 days, the package actually may contain up to 20 daily dosages of fluoxetine, or a pharmaceutically acceptable salt thereof, and 12 to 20 daily dosages of placebo, and even more preferably about 15 to about 18 daily dosages of fluoxetine, or a pharmaceutically acceptable salt thereof, and about 13 daily dosages of placebo. In this embodiment the female would start the placebo dosages on the first day of menses or the subsequent day thereof and then start the pharmaceutical after all 13 daily placebo dosages were completed. If the onset of menses occurs before all of the daily dosages of fluoxetine, or pharmaceutically acceptable salt thereof, in the package are used, then the female will discard that package and may repeat the treatment method by beginning the administration of a placebo from a new package. If the female has a longer than average menstrual cycle she may take the additional daily dosages of fluoxetine, or pharmaceutically acceptable salts thereof, i.e., up to 18 days, contained in the package until either the additional dosages are exhausted or menses begins.

The package of this invention, requires no indicia to determine the placebo dosage period based on the number of days in the female's menstrual cycle. Instead the dosage of both the placebo and fluoxetine or pharmaceutical acceptable salt thereof is set and the female simply starts with the placebo as described above. These embodiments may be used to treat premenstrual syndrome, including its more severe form, i.e., premenstrual dysphoric disorder.

This invention is also directed to a package for a non-continuous administration of a pharmaceutical to a human female for a condition associated with the female's menstrual cycle in accordance with the method of this invention. The package comprises an allotment of daily placebo dosages and an allotment of daily pharmaceutical dosages. No means of determining how many days of placebo dosage must be administered during the menstrual cycle are required and/or present since the user takes all the placebo present no matter what the length of the menstrual cycle. The package may take any shape or form. The female is simply instructed to take all the placebo provided in the package on a daily basis and upon completion of the placebo begins the daily administration of the pharmaceutical.

In another preferred specific embodiment of the package of the present invention the daily placebo dosages and the daily pharmaceutical dosages are single dosage units. In a more specific embodiment the single dosage unit is in a form selected from a tablet, a capsule and a pulvule. In a more preferable specific embodiment, wherein the pharmaceutical dosage is fluoxetine, or a pharmaceutically acceptable salt thereof, the single dosage unit is in pulvule form.

The package of this invention will contain an allotment of daily placebo dosage and an allotment of daily pharmaceutical dosage to practice the method of this invention. Most preferably the allotment of daily pharmaceutical dosage will include fluoxetine or a pharmaceutically acceptable salt thereof. In a particularly preferred embodiment the package will be a card design having the allotment of daily placebo and daily pharmaceutical dosage disposed thereon. Preferably each dosage may be individually removed from the package such as in a blister pack type package. Instructions either on the package or accompanying this preferred package may instruct a female to begin administration of the daily placebo dosage on the first or subsequent days of her menstrual cycle. The user may also be instructed to continue daily administration of each dosage in sequential order until the first of either the pharmaceutical dosages are exhausted or the start of menses. In that regard it should be noted that the packages may be designed to include extra pharmaceutical dosages with instructions to administer those extra dosages if, for example, the first fifteen pharmaceutical dosages are completed prior to the onset of menses. The user is preferably instructed to discard the package once menses begins and start a new package.

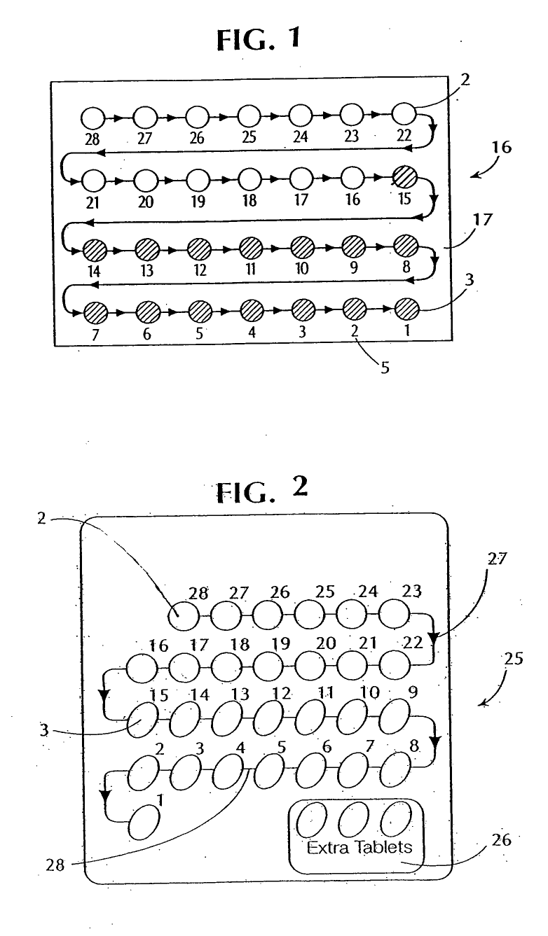

FIG. 1 is an illustration of exemplary package 16 of the present invention. The package includes a total of twenty eight dosages, wherein thirteen are daily placebo dosages 2 and fifteen are daily pharmaceutical dosages 3. Both the placebo and pharmaceutical dosages are disposed on a rectangular base card 17, where they are held by any suitable means, e.g. blister packaging. The base card includes numbers 5 corresponding to the length of the average female's menstrual cycle. A female would start administration with the placebo dosage, taking one dosage daily, counting down on the package to the next lowest number each day. Once the last pharmaceutical dosage is administered the package is discarded and administration begins from a new package, or, if a new menstrual cycle begins before the last pharmaceutical dosage is administered, then the package is discarded and administration begins from a new package.

Another embodiment of this invention is illustrated in FIG. 2. Package 25 is a card containing 15 pharmaceutical dosages 3 in numbered order and 13 placebo dosages 2 in numbered order. It should be readily apparent that the number of pharmaceutical dosages and placebo dosages could be altered. The package 25 also contains three extra pharmaceutical dosages 3 in a separate area 26. The card includes numbers 5 from twenty-eight to one, which are associated with the placebo dosages 2 and the pharmaceutical dosages 3. Thereafter, administration proceeds on a daily basis to the dosage associated with the next lowest number in the direction of the arrows 27 following pathway 28. When the last pharmaceutical dosage 3 along pathway 28 is administered the user may optionally begin administration of the pharmaceutical dosage 3 contained in the extra dosage area 26 until either the pharmaceutical dosage 3 in the extra dosage area 26 is exhausted or the onset of menses occurs. When either the last pharmaceutical dosage is administered, or if a new menstrual cycle begins before the last pharmaceutical dosage is used then the package is discarded. Administration from a new package may begin when a new menstrual cycle starts.

The packages illustrated in FIGS. 1-2 could readily be altered to hold different amounts of the pharmaceutical dosage and the placebo dosage. Of course, alternate embodiments of all of these packages can be readily envisioned.

1. A method of non-continuous administration of a pharmaceutical to a human female for a condition associated with the luteal phase of a female's menstrual cycle, comprising the steps of:
a) orally administering a daily placebo dosage, starting on the first day of the female's menstrual cycle or the subsequent day thereof and continuing the daily oral administration of the daily placebo dosage for a placebo dosage period, and
b) orally administering a daily dosage of the pharmaceutical starting on the day after the last daily placebo dosage was administered and continuing the daily oral administration of the daily dosage of the pharmaceutical for the earlier of (i) a pharmaceutical dosage period or (ii) the onset of menses,
wherein the pharmaceutical dosage period is an effective number of days to treat the condition in a range from 5 to 21 days and the placebo dosage period is in a range from 7 to 23 days.

2. The method of claim 1, wherein said condition is selected from the group consisting of premenstrual syndrome, migraine headache, endometriosis, psoriasis, acne, dysmenorrhea, neurosia, and premenstrual cramps.

3. The method of claim 2, wherein said condition is premenstrual syndrome.

4. The method of claim 3, wherein said pharmaceutical is a selective serotonin reuptake inhibitor.

5. The method of claim 4, wherein said selective serotonin reuptake inhibitor is selected from the group consisting of citalopram, escitalopram, fluvoxamine, paroxetine, fluoxetine, sertraline and pharmaceutically acceptable salts thereof.

6. The method of claim 5, wherein said selective serotonin reuptake inhibitor is fluoxetine or a pharmaceutically acceptable salt thereof.

7. The method of claim 6, wherein the selected number of days to administer said fluoxetine, or the pharmaceutically acceptable salt thereof, during the pharmaceutical dosage period is in a range of 15 to 18 days.

8. The method of claim 1, wherein said method is conducted without calculating the number of placebo dosages that should be administered based on an ascertained length of the female's menstrual cycle.

9. The method of claim 7, wherein said pharmaceutical dosage period is 15 days.

10. The method of claim 9, wherein the placebo dosage period is 13 days.

11. The method of claim 3, wherein said premenstrual syndrome is premenstrual dysphoric disorder.

12. The method of claim 1, wherein each placebo dosage and pharmaceutical dosage is contained in a single dosage unit.

13. The method of claim 1, wherein said pharmaceutical is selected from the group consisting of a selective serotonin reuptake inhibitor, an analgesic, a diuretic, and cortico steroids.

14. The method of claim 1, wherein a total number of days to administer said pharmaceutical and said placebo is 28 days.

15. The method of claim 1, wherein steps a and b are repeated at the onset of menses.

16. A method of non-continuous administration of fluoxetine or a pharmaceutically acceptable salt thereof to a human female for the treatment of pre-menstrual syndrome, comprising the steps of:
a) orally administering a daily placebo dosage, starting on the first day of the female's menstrual cycle or the subsequent day thereof and continuing the daily oral administration of the daily placebo dosage for a placebo dosage period of 13 days, and
b) orally administering a daily dosage of fluoxetine or a pharmaceutically acceptable salt thereof, starting on the day after the last daily placebo dosage was administered and continuing the daily oral administration of the daily dosage of fluoxetine or the pharmaceutically acceptable salt thereof for the earlier of (i) 15 to 18 days or (ii) the onset of menses.

17. The method of claim 16, wherein the method is conducted without calculating the number of placebo dosages that should be administering based on an ascertained length of the female's menstrual cycle.

18. A package for delivering a non-continuous administration of fluoxetine or a pharmaceutically acceptable salt thereof to a human female for the treatment of pre-menstrual syndrome, said packaging comprising:
an allotment of daily placebo dosages and an allotment of daily dosages of fluoxetine or pharmaceutically acceptable salts thereof, the daily placebo dosages and the daily dosages of fluoxetine or pharmaceutically acceptable salts thereof arranged in the package for administration according to the method of claim 16, wherein the package is free of means to calculate the number of placebo dosages that should be administered based on an ascertained length of the female's menstrual cycle.
